(19) United States
(12) Patent Application Publication
(10) Pub. No.: US 2019/0248717 A1
(43) Pub. Date: Aug. 15, 2019

(54) OXIDATIVE DEHYDROGENATION OF ALKANES TO ALKENES, AND RELATED SYSTEM

(71) Applicant: Battelle Energy Alliance, LLC, Idaho Falls, ID (US)

(72) Inventors: Anne M. Gaffney, Idaho Falls, ID (US); Daniel M. Ginosar, Idaho Falls, ID (US); Olivia M. Mason, Riverdale, NY (US); Gennaro J. Maffia, Newton Square, PA (US)

(21) Appl. No.: 16/345,339

(22) PCT Filed: Nov. 3, 2017
(86) PCT No.: PCT/US17/59844

§ 371 Date: Apr. 26, 2019

Related U.S. Application Data
(60) Provisional application No. 62/416,801, filed on Nov. 3, 2016.

(51) Int. Cl. C07C 5/48; B01D 71/02; B01D 3/14; B01J 27/057; B01D 53/22; C07C 7/144; C07C 7/04; C07C 7/00; B01J 19/00; B01J 19/24; B01J 8/02; B01J 8/18

(52) U.S. Cl. CPC C07C 5/48; B01D 71/022; B01D 3/145; B01J 27/0576; B01D 53/228; C07C 7/144; B01J 2219/00074; C07C 7/005; B01J 19/0013; B01J 19/2415; B01J 8/0278; B01J 8/1836; B01J 2208/00106; C07C 7/04

(57) ABSTRACT

A method of producing and separating an alkene, such as ethylene, from an alkane, such as ethane. The method comprises subjecting a feedstock comprising ethane to oxidative dehydrogenation to produce an ethylene stream. The ethylene stream is passed through a membrane separation unit to separate the ethylene from unreacted ethane in the ethylene stream. The ethylene is recovered from the membrane separation unit. A system configured to produce ethylene is also disclosed. The system comprises at least one ODH reactor, a heat management unit coupled to the at least one ODH reactor, and at least one membrane separation unit comprising at least one membrane. The ODH reactor is configured to convert ethane to ethylene. The heat management unit is configured to reduce a temperature of the ethylene. The at least one membrane is configured to separate the ethylene from unreacted ethane.

### PRIORITY CLAIM

This application is a national phase entry under 35 U.S.C. § 371 of International Patent Application PCT/US2017/059844, filed Nov. 3, 2017, designating the United States of America and published in English as International Patent Publication WO 2018/085614 A1 on May 11, 2018, which claims the benefit of the filing date under Article 8 of the Patent Cooperation Treaty of United States Provisional Patent Application Ser. No. 62/416,801, filed Nov. 3, 2016, for “OXIDATIVE DEHYDROGENATION OF ALKANES TO ALKENES, AND RELATED SYSTEMS.”

### STATEMENT REGARDING FEDERALLY SPONSORED RESEARCH OR DEVELOPMENT

This invention was made with government support under Contract Number DE-AC07-051D14517 awarded by the United States Department of Energy. The government has certain rights in the invention.

### TECHNICAL FIELD

The disclosure, in various embodiments, relates generally to alkene production from an alkane. More specifically, the disclosure, in various embodiments, relates to the production of ethylene and other alkenes from ethane and other alkanes by oxidative dehydrogenation (ODH) and purification of the alkene by membrane separation.

### BACKGROUND

The nation's goal to increase energy efficiency and reduce the carbon footprint calls upon many industries to relook at the way we produce and consume energy. The oil and gas industry has practiced thermal cracking of petroleum for the past 100 years. The thermal cracking of petroleum fractions produces an extensive list of compounds, including ethylene and other alkenes and diolefins that are utilized across the petrochemical industry. For instance, ethylene is used to produce polyethylene, which is a plastic material used in a variety of products, ethylene oxide, ethylene dichloride, and ethylbenzene. The global capacity of ethylene is expected to reach 179 MTPA by 2018.

Ethylene is conventionally produced from ethane by steam pyrolysis or steam cracking, which systems have hundreds of individual pieces of equipment and more than twenty sections, some containing many unit operations. These sections of the systems are used to recover and purify major products of the ethane to ethylene reaction. Producing plants to include the many sections is capital intensive and costly to operate. The thermal process of converting the ethane to ethylene achieves an ethylene selectivity of 80-85% at an ethane conversion of 55-65%. The process is endothermic and is conducted at a temperature between 800° C.-900° C., making the process energy intensive and costly.

Oxidative dehydrogenation (ODH) has been investigated as an alternative for producing ethylene from ethane. The ethane is reacted with an oxidizing agent in the presence of a catalyst to produce the ethylene. The ODH of ethane is exothermic and, therefore, the ODH reaction is more energy efficient than the steam cracking process. If the ODH reaction is quenched quickly, byproduct production is minimal. However, the ethylene conversion, ethane selectivity, and ethylene yield are highly dependent on the catalyst used and reaction conditions. As disclosed in U.S. Patent Application Publication 2005/0124840, the ethylene is separated from combustible, reactive, or non-reactive byproducts by cryogenic separation, distillation, or membrane separation. However, cryogenic separation and distillation are expensive and energy-intensive techniques. Additionally, the membrane separation is not selective and only separates the ethylene from the combustible, reactive, or non-reactive byproducts.

### BRIEF SUMMARY

An embodiment of the disclosure comprises a method of producing ethylene. The method comprises subjecting a feedstock comprising ethane to oxidative dehydrogenation to produce an ethylene stream. The ethylene stream is passed through a membrane separation unit to separate the ethylene from unreacted ethane in the ethylene stream. The ethylene is recovered from the membrane separation unit.

Another embodiment of the disclosures comprises a method of producing ethylene. The method comprises contacting ethane and oxygen in the presence of a mixed metal oxide catalyst to produce a stream comprising ethylene. The stream is passed through a membrane separation unit to separate the ethylene from the stream. Ethylene having an ethylene content of at least about 90% is recovered from the stream.

Yet another embodiment of the disclosure comprises a system configured to produce ethylene. The system comprises at least one ODH reactor configured to convert ethane to ethylene. A heat management unit is coupled to the at least one ODH reactor and configured to reduce a temperature of the ethylene. At least one membrane separation unit comprising at least one membrane is configured to separate the ethylene from unreacted ethane.

### BRIEF DESCRIPTION OF THE DRAWINGS

FIG. 1 is a schematic drawing illustrating a process scheme and system for producing ethylene according to embodiments of the disclosure;

FIG. 2 is a schematic drawing illustrating a process scheme and system for producing ethylene according to embodiments of the disclosure;

FIG. 3 is a schematic drawing illustrating a process scheme and system for producing ethylene according to embodiments of the disclosure; and

FIG. 4 is a schematic drawing illustrating a membrane separation unit for producing ethylene according to embodiments of the disclosure.

### DETAILED DESCRIPTION

A method of producing and separating an alkene, such as ethylene, from an alkane, such as ethane, is disclosed, as is a system configured to produce the alkene from the alkane. The system includes an ODH reactor coupled with a heat management unit and a membrane separation unit. No alkene fractionator, such as a distillatory, is utilized in the system to recover the alkene. Instead, the membrane separation unit is utilized to recover the alkene. The method and system improve the production of the alkene, and provide an increase in energy efficiency, a reduction and elimination of byproducts and waste streams, and a modular cost-advantageous approach to producing the alkene. The method and system according to embodiments of the disclosure lower the carbon footprint compared to conventional methods and conventional systems of producing, such as by steam cracking, and separating the alkene, such as by compression and cryogenic distillation. The carbon footprint of the method and system according to embodiments of the disclosure may be reduced by about 30% or more. The method and system according to embodiments of the disclosure may reduce energy consumption by from about 50% to about 75% compared to conventional systems and methods of producing and separating the alkene. The method and system according to embodiments of the disclosure may also exhibit reduced capital and operating costs than conventional methods and systems. By increasing the purity of the alkene exiting the ODH reactor and improving the efficiency of separating the alkene from the alkane, the alkene of a chemical grade or of a polymer grade may be achieved. The method and system according to embodiments of the disclosure may achieve zero energy per pound (or kilogram) of ethylene produced and zero carbon footprint.

The conversion of ethane to ethylene and the separation of the ethylene provides the greatest amounts of energy production and carbon savings and, therefore, is described in detail herein. However, embodiments of the disclosure may be used to convert other alkanes to their respective alkenes and alkenes to diolefins, and to separate the alkenes and diolefins, such as the conversion of propane to propene, butane to butene, etc.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof. As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

As used herein, the term “alkane” means and includes a saturated, straight, branched, or cyclic hydrocarbon containing from two carbon atoms to eight carbon atoms. Examples include, but are not limited to, ethane, propane (n-propane, isopropane, cyclopropane), butane (n-butane, isobutane, sec-butane, tert-butane, cyclobutane), pentane (n-pentane, tert-pentane, neopentane, isopentane, sec-pentane, 3-pentane, cyclopentane), hexane (isohexane, cyclohexane, 3-methylpentane, 2,2-dimethylbutane, 2,3-dimethylbutane), heptane (n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane), or octane (n-octane, 2-methylheptane, 3-methylheptane, 4-methylheptane, 3-ethylhexane, 2,2-dimethylhexane, 2,3-dimethylhexane, 2,4-dimethylhexane, 2,5-dimethylhexane, 3,3-dimethylhexane, 3,4-dimethylhexane, 3-ethyl-2-methylpentane, 3-ethyl-3-methylpentane, 2,2,3- trimethylpentane, 2,2,4-trimethylpentane, 2,3,3-trimethylpentane, 2,3,4-trimethylpentane, 2,2,3,3-tetramethylbutane, ethylbenzene).

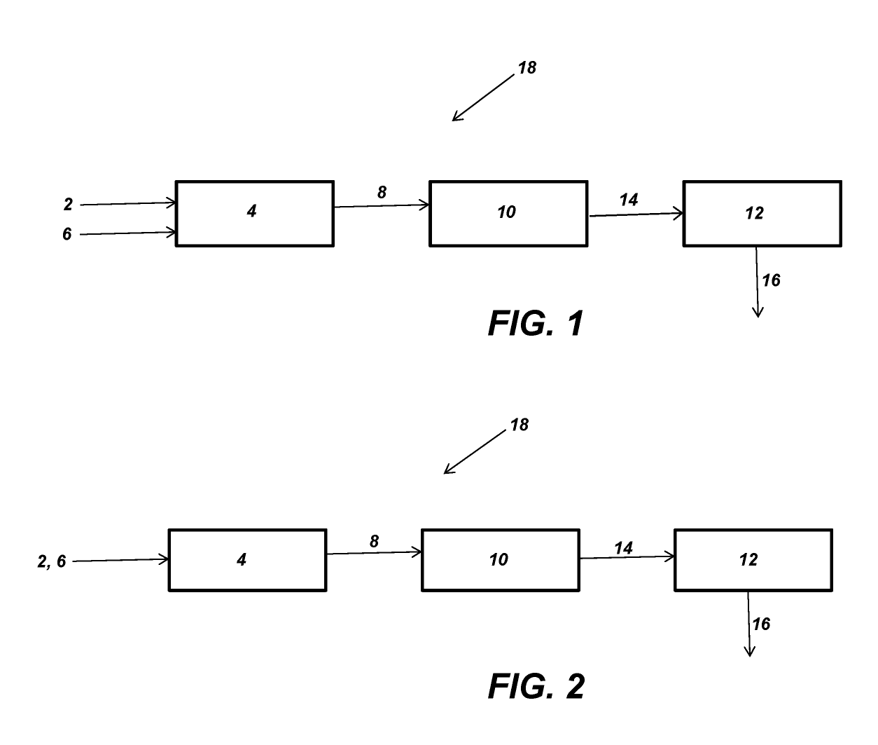

To convert the ethane to ethylene, an ethane feedstock 2 may be subjected to oxidative dehydrogenation in the presence of an oxidizing agent and a catalyst, as shown in FIG. 1. The ethane feedstock 2 may be introduced into the ODH reactor 4 for the oxidative dehydrogenation. The ethane feedstock 2 may include an excess of two carbon (C₂) compounds including, but not limited to, ethane. The ethane feedstock 2 may include at least about 50% by volume of ethane, such as from about 50% by volume to about 95% by volume of ethane. The ethane content may depend on the origin of the ethane feedstock 2, such as ethane from natural gas, ethane from oil shale, or an ethane byproduct from a pyrolysis plant. The ethane feedstock 2 may be obtained from any source. Impurities in the ethane feedstock 2 may be removed before the ODH reaction to prevent or reduce poisoning of the catalyst or the production of undesirable byproducts, such as carbon oxides, other hydrocarbons, etc., that may be difficult to remove. For other alkane to alkene conversions, the feedstock may include an excess of C₂+ compounds, such as compounds having between two carbon atoms and eight carbon atoms, such as a combination of one or more of C₂ compounds, C₃ compounds, C₄ compounds, C₅ compounds, C₆ compounds, C₇ compounds, or C₈ compounds.

The ethane feedstock 2 may be introduced into the ODH reactor 4 with an optional diluent (not shown) to minimize the production of exotherms during the ODH reaction. The diluent may be water (e.g., steam), nitrogen, carbon dioxide, or other inert gas to reduce flammability of the reactants in the ODH reactor 4. Ethylene produced by the method may also be used as the diluent since the ethylene, once produced, does not decompose or undergo secondary reactions.

The oxidizing agent 6 may include, but is not limited to, oxygen (O₂), air, carbon dioxide (CO₂), nitrous oxide, ozone, or combinations thereof. In one embodiment, the oxidizing agent 6 is O₂. However, other oxidizing agents may be used. The oxidizing agent 6 may be combined with an inert gas, such as nitrogen, helium, or argon, to reduce the flammability of the oxidizing agent during the ODH reaction. The oxidizing agent may be the limiting reactant in the ODH reaction.

The catalyst may be a heterogeneous catalyst, such as a mixed metal oxide (MMO) catalyst selected to provide a high ethane conversion, such as greater than about 80 mole %, greater than about 85 mole %, or greater than about 90 mole %. The MMO catalyst may also be selected to provide a high ethylene selectivity at all ethane conversion levels, such as greater than about 90 mole %. The particle size (e.g., spherical diameter) of the MMO catalyst may be between about 0.1 cm and about 0.5 cm. The MMO catalyst may include at least one of molybdenum (Mo) atoms, vanadium (V) atoms, niobium (Nb) atoms, tellurium (Te) atoms, antimony (Sb) atoms, and oxygen (O) atoms. In one embodiment, the MMO catalyst is a MoₐV_{b}Nb_{c}Te_{d}SbₑO_{f} catalyst, where a is 1, b is a real number between about 0.01 and about 1, c is a real number between about 0.001 and about 1, d is a real number between about 0.01 and about 1, e is a real number between about 0.001 and about 1, and f is dependent on the oxidation state(s) of the other elements, such as between about 3.0 and about 4.7. However, other MMO catalysts may be used. The MMO catalyst may be produced by conventional techniques, which are not described in detail herein. For instance, the MMO catalyst may be produced as described in U.S. Pat. Nos. 8,519,210 and 9,169,442 and U.S. Patent Application Publication 2010/0256432. The MMO catalyst may be housed within the ODH reactor 4, such as within a bed of the ODH reactor 4.

The ethane feedstock 2 and the oxygen 6 may be introduced into the ODH reactor 4, which is maintained at temperature and pressure conditions suitable for the oxidative dehydrogenation reaction. The ethane of the ethane feedstock 2 and the oxygen 6 are reacted in the vapor phase. As shown in FIG. 1, the ethane feedstock 2 and the oxygen 6 may be introduced into the ODH reactor 4 in one or more separate streams. Alternatively, the ethane feedstock 2 and the oxygen 6 may be combined and introduced into the ODH reactor 4 as a single stream, as shown in FIG. 2. The ethane feedstock 2 and the oxygen 6 may be heated to temperature before their introduction into the ODH reactor 4 or the ethane feedstock 2 and the oxygen 6 may be brought to the desired temperature within the ODH reactor 4.

The ethane feedstock 2 and the oxygen 6 may be introduced into the ODH reactor 4 and reacted at a temperature of from about 150° C. to about 500° C., such as from about 300° C. to about 450° C. An inlet pressure of the ODH reactor 4 may range from about 0.1 bar (about 0.01 megapascal) to about 30 bar (about 3 megapascals), such as from about 0.1 bar (about 0.01 megapascal) to about 20 bar (about 2 megapascals). In one embodiment, the inlet pressure of the ODH reactor 4 may be within a range from about 4 bar (about 0.4 megapascal) to about 7 bar (about 0.7 megapascal). The ethane feedstock 2 and the oxygen 6 may be flowed over the MMO catalyst at the reaction temperature and pressure for a residence time of from about 0.1 second to about 15 seconds, such as from about 5 seconds to about 15 seconds. The ethane feedstock 2 and the oxygen 6 may be passed over the MMO catalyst in the ODH reactor 4. The ethane of the ethane feedstock 2 is converted to ethylene in nearly stoichiometric amounts, with water and small amounts of carbon oxides (carbon monoxide (CO) and/or carbon dioxide (CO₂)) also produced according to the following reaction:

C₂H₆+0.55 O₂→0.98 C₂H₄+0.03 CO₂+0.005 C₂H₄O₂+1.03 H₂O

Acetic acid, acetylene, or combinations thereof may also be produced depending on the reaction conditions and catalyst used. The ethane in the ethane feedstock 2 is converted to ethylene with near stoichiometric yields at about 10% conversion. The rate of the ODH reaction may depend on the configuration of the ODH reactor 4, the partial pressure of ethane, and frictional pressure drop when multiple ODH reactors 4 are used in the system 18.

To produce the ethylene at a high selectivity, an excess of ethane and limited oxygen may be provided to the ODH reactor 4. The ethane feedstock 2 may account for about 95% of the volume introduced into the ODH reactor 4 and the oxygen 6 may account for about 5% of the volume. Thus, the ethane is supplied in excess, and the oxygen is limiting. By using excess ethane, condensing and recycling the diluent, such as water, is not needed. In addition, the excess of ethane maintains the ethane concentration in the ODH reactor 4 at well above its explosive limit. The excess of ethane also moderates exotherms and enables heat recovery and temperature management in the system.

The ODH reaction is exothermic and produces large amounts of heat. The ODH reactor 4 may be maintained at near isothermal conditions by removing the heat, as described in more detail below. The heat produced by the ODH reaction may be captured and integrated into the method of producing the ethylene, increasing the energy efficiency of the ethylene production. In contrast, conventional methods of producing ethylene are endothermic so recovering and using the heat in the conventional methods is not possible.

The ODH reaction results in conversion of the ethane to ethylene at an ethylene conversion of greater than about 90%, with a greater than about 95% selectivity to ethylene. The reaction may result in a conversion and selectivity of about 98%. Once the ethylene is produced, no secondary reactions will occur since ethylene is refractory to the MMO catalyst. Thus, the system 18 may include multiple ODH reactors without loss of selectivity in the ODH reaction, as described in more detail below.

Once produced, the ethylene may exit the ODH reactor in an ethylene stream 8, i.e., the reactor effluent, which is cooled and separated, such as with a membrane, to recover the purified ethylene 16. The produced alkene, such as the ethylene, need not be immediately removed upon conversion of the alkane because there are not expected to be any significant secondary reactions. As described in more detail below, the ethylene stream 8 may be introduced to the heat management unit 10 to reduce the temperature of the ethylene stream 8. Depending on the temperature of the ethylene stream 8 exiting the ODH reactor 4, the ethylene stream 8 may be cooled before introduction into the membrane separation unit 12, as described in more detail below. The cooled ethylene stream 14 may be introduced to the membrane separation unit 12 to separate the ethylene from other components, such as unreacted ethane and the minimal by-products, of the ethylene stream 8. The ethylene may be removed by passing the ethylene stream 8 through at least one membrane 20 of the membrane separation unit 12 (see FIG. 4). As described in more detail below, the ethylene may be selectively transported through the membrane 20 while the other components, such as the unreacted ethane, do not pass through the membrane 20.

The purified ethylene 16 recovered from the membrane separation unit 12 may be enriched in ethylene compared to that of the ethylene stream 8. By way of example only, the purified ethylene 16 recovered from the membrane separation unit 12 may be at a purity suitable for commercial purposes, such as commercial grade ethylene. The purified ethylene 16 recovered from the membrane separation unit 12 may be a chemical grade ethylene, such as having an ethylene content of at least about 90%. Alternatively, the purified ethylene 16 recovered from the membrane separation unit 12 may be a polymer grade ethylene, such as having an ethylene content of at least about 99%, such as about 99.5% or greater or about 99.95% or greater.

Depending on the reaction conditions and catalyst used, the ethylene stream 8 may also include water, unreacted ethane, CO, CO₂, acetylene, acetic acid, or combinations thereof. The ethylene stream 8 may be cooled and the components removed by passing the cooled ethylene stream 14 over a sorbent (not shown) or by chemically converting the components to other compounds that may be easily removed from the ethylene stream 8. By way of example only, any carbon dioxide in the ethylene stream 8 may be removed by absorption, such as amine absorption using an alkanolamine, adsorption, or other conventional techniques. Any carbon monoxide in the ethylene stream 8 may be removed by cooling the ethylene stream 8 to a temperature of less than or equal to about 100° C., converting the carbon monoxide to carbon dioxide, and then removing the carbon dioxide by amine absorption. Other conventional techniques for carbon monoxide recovery may also be employed. Any water in the ethylene stream 8 may be removed by cooling, condensing, and drying. Any acetylene in the ethylene stream 8 may be reacted with hydrogen to form ethylene, ethane, or combinations thereof. The ethylene produced from the acetylene may be combined with the ethylene stream 8, while the ethane produced from the acetylene is recycled and combined with the ethane feedstock 2. The reaction of the acetylene is exothermic, heterogeneously catalyzed, and has an inlet temperature at the start of run of the catalyst of about 100° C. An exotherm of from about 30° C. to about 50° C. is allowed and then the material is removed from the ODH reactor 4 and cooled externally. The cooling may occur using a very low pressure boiler feed water so that the steam is produced and recovered, or cooling water in which case the heat of reaction is not recovered.

The unreacted ethane in the ethylene stream 8 may be removed by passing the ethylene stream 8 through the membrane 20 of the membrane separation unit 12. As described in more detail below, the ethylene may be selectively transported through the membrane 20 while the ethane does not pass through the membrane 20. The ethane may be periodically recovered from the membrane separation unit 12 and combined with the ethane feedstock 2 to convert the ethane to additional ethylene.

A system 18 for producing the ethylene from ethane includes at least one ODH reactor 4, the heat management unit 10, and the membrane separation unit 12. While the heat management unit 10 is illustrated in FIGS. 1-3 as being a separate component from the ODH reactor 4, the heat management unit 10 may be part of the ODH reactor 4, such as including a coolant in the ODH reactor 4. Compared to conventional systems having twenty or more sections, the system 18 according to embodiments of the disclosure includes much fewer sections, with an optional distillator after the membrane separation to achieve the desired purity of the ethylene. Thus, the system 18 according to embodiments of the disclosure is significantly less complicated and less costly than conventional systems.

The ODH reactor 4 may be configured to receive and react the ethane feedstock 2 and the oxygen 6 in the vapor phase. The ODH reactor 4 contains the MMO catalyst configured in a bed, such as a fixed bed, a fluidized bed, a circulating bed, or a packed bed (i.e., a plug flow reactor). Conventional ODH reactors are known in the art and, therefore, are not described in detail herein. The ODH reactor 4 may include, but is not limited to, as tubular ODH reactor, a fixed bed ODH reactor, a reverse flow catalytic ODH reactor, a plate and frame ODH reactor, a fluidized bed ODH reactor, a packed bed ODH reactor, or a circulating bed ODH reactor. In one embodiment, the ODH reactor 4 is a tubular ODH reactor that includes an elongated reaction vessel configured to enable the ethane feedstock 2 to flow into, through, and out of the reaction vessel through apertures located proximal to opposing ends of the reaction vessel. The tubular ODH reactor may be a shell and tube reactor, where the shell has a coolant i.e., a heat transfer fluid, circulating within the shell or heat removal tubes within the catalyst bed. In one embodiment, the MMO catalyst is contained within the tubes of the tubular ODH reactor and the coolant, such as supercritical water, compressed water, a heat transfer fluid, or a molten salt, is circulated through the shell of the tubular ODH reactor. By way of example only, high volumes of ethylene may be produced in a tubular ODH reactor 4 containing 20,000 tubes or more, where the tubes are packed tubes including a silver catalyst.

The ODH reactor 4 includes at least one inlet (not shown) for introducing the ethane feedstock 2 and the oxygen 6 and at least one outlet (not shown) for removing the ethylene stream 8. The ethane feedstock 2 and the oxygen 6 may, alternatively, be combined external to the ODH reactor 4 and introduced into the ODH reactor 4 as a single stream through the inlet, as shown in FIG. 2. Multiple inlets (not shown) may be present if the ethane feedstock 2 and the oxygen 6 are to be introduced into the ODH reactor 4 as separate streams, as shown in FIG. 1. Alternatively, the ethane feedstock 2 and/or the oxygen 6 may be introduced to the ODH reactor 4 in portions, such as a portion at the inlet of the ODH reactor 4, a portion at a midpoint of the ODH reactor 4, etc.

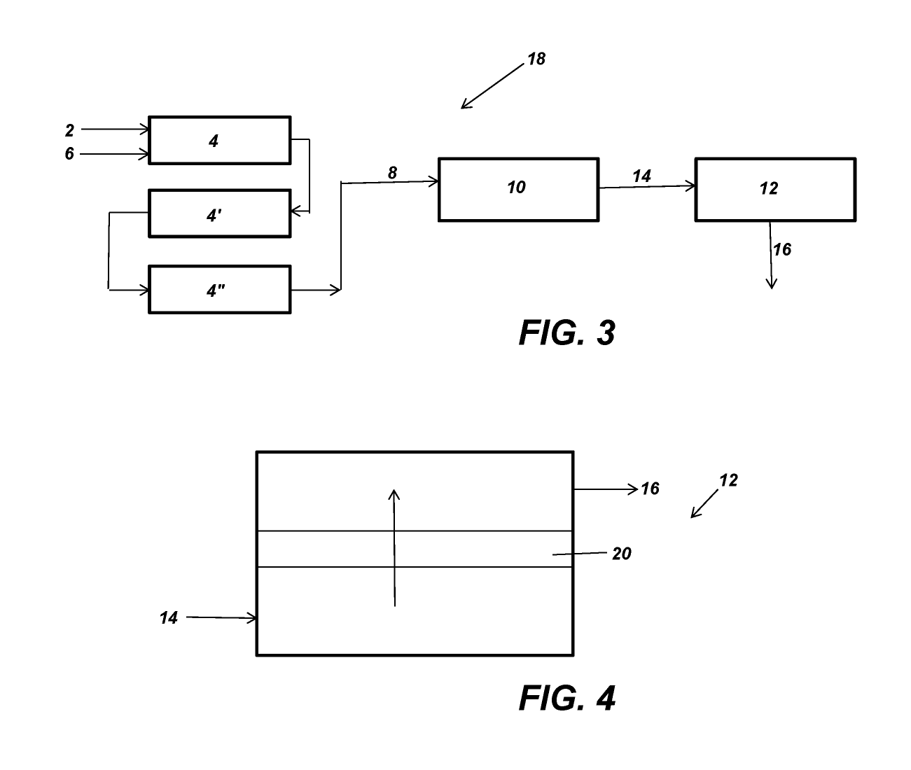

To increase efficiency, two or more ODH reactors 4 connected in series may be used in the system 18, as shown in FIG. 3. The ODH reactors 4 may be connected by conventional techniques, such as by tubing, valves, etc., which are not described in detail herein. While FIG. 3 illustrates three ODH reactors 4, 4′, 4″, the number of ODH reactors 4 may be increased or decreased depending on the composition of the ethane feedstock 2 to be processed. The number of ODH reactors 4 in the system 18 may also affect the costs of the system 18. By reducing the number of ODH reactors 4 relative to conventional systems, the capital and operating costs of the system according to embodiments of the disclosure may be reduced. Each of the ODH reactors 4, 4′, 4″ may have one of the configurations previously described. Each of the ODH reactors 4, 4′, 4″ may have the same configuration or a different configuration. By way of example only, the ODH reactors 4, 4′, 4″ may include packed bed ODH reactors, packed tubular ODH reactors, or combinations thereof configured in series. The ethane feedstock 2 and the oxygen 6 may be introduced to a first ODH reactor 4 of the ODH reactors 4, 4′, 4″ with no further additions of the ethane feedstock 2 and the oxygen 6 to the system 18. Alternatively, the ethane feedstock 2 and the oxygen 6 may be introduced to the first ODH reactor 4, and additional portions of the ethane feedstock 2 and/or the oxygen 6 added to a second ODH reactor 4′ or a third ODH reactor 4″ of the system 18. Since the ODH reaction is run as oxygen limiting, the oxygen 6 may be introduced to the ODH reactors 4, 4′, 4″ in multiple portions. The further additions of oxygen 6 to the ODH reactors 4, 4′, 4″ may be needed to maintain the ethane concentration above its explosive limit. Since no secondary reactions occur once the ethylene is produced, the system 18 may include multiple ODH reactors 4, 4′, 4″ without loss of selectivity in the ODH reaction.

If the ODH reactor 4 includes multiple adiabatic packed bed ODH reactors 4 in series, the ethane conversion may initially be 10% with oxygen limiting the ODH reaction. The amount of oxygen will be equivalent in each of the beds, about 10% of the original total, resulting in about 10% of the original ethane to be converted in each of the beds. Temperature exotherms will be about 100° C. for each of the packed beds, with an equivalent amount of cooling utilized between each of the beds. Without being bound by any theory, it is believed that the resulting axial temperature profile of the system 18 is such that the temperature will appear as a saw tooth with the net temperature rises occurring during the exothermic reaction. The temperature drop occurs during the direct quench or indirect cooling during external shell and tube heat exchanger. Due to the pressure drop in the ODH reactors 4 and auxiliaries of the initial pieces of equipment, the rate of the ODH reaction may decrease in the latter ODH reactors 4. To increase the rate of the ODH reaction, higher reaction temperatures may be used. Alternatively, the ODH reaction may be conducted at a reduced conversion to maintain the reaction rate. However, the exotherms may remain essentially the same at 100° C., although the temperature of the ethylene stream 8 exiting the ODH reactors 4 will increase with each subsequent ODH reactor 4.

The tubes of the packed bed ODH reactors 4 may be about 4 m long and provide about 25% conversion. The system 18 may include 4 or 5 bundles of tubes with interstage injection of the oxygen. The residence time in each of the bundles may be about 5 seconds. The kinetics of the ODH reaction may be first order for the ethane and the oxygen, with the ethane provided in excess while the oxygen is limiting. As the pressure drops between the bundles of tubes, the rate of reaction decreases. To maintain the ethane conversion, the temperature may be increased by adjusting the temperature of the coolant. By the latter ODH reactors 4, the temperature may exceed 400° C. The coolant may be selected to operate at that temperature, such as by using supercritical water or a molten salt.

The ethane feedstock 2 and the oxygen 6 may be reacted in the ODH reactor 4, as previously described, to produce the ethylene stream 8. The ethylene stream 8 may be introduced into the heat management unit 10 to remove the heat generated by the exothermic ODH reaction. The heat management system 18 may be coupled to the ODH reactor 4 by conventional techniques, such as by tubing, valves, etc., which are not described in detail herein. The heat management system 18 is configured to remove the heat, preventing runaway temperatures in the oxygen environment of the ODH reactor 4 and enabling stable operation of the ODH reactor 4. The heat management system 18 may reduce the temperature of the ethylene stream 8 to a temperature sufficient to pass through the membrane separation unit 12 without degrading or otherwise affecting the membrane. The heat management system 18 may reduce the temperature of the ethylene stream 8 from its post-reaction temperature (about 350° C.) to about 100° C. or lower. In some embodiments, the heat is recovered from the heat management system 18 and used as an energy source, such as for steam generation or water preheat. The recovered heat may be stored or exported as energy. In other embodiments, the heat is recovered and recycled in the system 18. By way of example only, the recovered heat may be used to heat the ethane feedstock 2 and the oxygen 6 in the ODH reactor 4 to reaction temperature.

Due to the amount of heat generated by the ODH reaction, conventional cooling techniques may not be sufficient or feasible for use as the heat management unit 10. For instance, subcritical water, water at a cooler temperature, or boiler feed water steam may be insufficient for use in reducing the temperature within the ODH reactor 4.

The heat management system 18 may be a supercritical water system (SCWS), such as that used in a nuclear power plant. Such supercritical water systems are known in the art and, therefore, are not described in detail herein. The SCWS may have a high thermal efficiency and include circulating water pumps that pressurize the water to above its critical pressure (P_{crit}). When the supercritical water is fed to the shell side of the ODH reactor 4, the water may be heated while serving as coolant. After capturing the heat from the ODH reactor 4, the supercritical water is let down through a turbine in a modified Rankine Cycle. The efficiency of the system 18 may approach about 50% in such cycles, thus improving the energy recovery and the maintenance of the energy availability level.

If the ODH reactor 4 is a multi-tube shell and tube reactor, the heat may be removed by the coolant circulating within the shell or within heat removal tubes within the catalyst bed. The coolant may be supercritical water, a heat transfer fluid, or a molten salt. The coolant may circulate through the shell of the ODH reactor 4, reducing and controlling the temperature within the ODH reactor 4 that is generated by the exothermic reaction. To avoid runaway conditions in the ODH reactor 4, exotherms may be kept to a maximum of from about 30° C. to about 50° C. The term “exotherm” means and includes a difference between a temperature of the ethane feedstock 2 at a certain axial position in the ODH reactor 4 and a temperature of the coolant at that same position.

If the ODH reactor 4 is a packed tubular ODH reactor, the heat may be removed and recovered by circulating the coolant in the shell side. The heat may be used directly to manufacture steam, similar to systems employed in SCWS in nuclear plants. The coolant may be a fluid other than high pressure water converting to steam because of the proximity to the critical conditions. Due to the amount of heat generated by the ODH reaction, it is unlikely that cooler water, such as subcritical water, and the corresponding steam generated may be used in the system 18. However, the steam generated may be used as an energy source in other systems.

If the system 18 includes multiple ODH reactors 4 in series, the heat management system 18 may include direct or indirect interbed cooling of the ODH reactors 4. The direct cooling may include supplying a portion of the ethane feedstock 2 to the ODH reactor 4 at a lower temperature than the reaction temperature, in a so-called “cold shot cooling,” which is a low cost heat and effective way to manage exotherms in the ODH reactor 4. The cooled portion (not shown) of the ethane feedstock 2 may be introduced into the ODH reactors 4 at various axial positions along the bed of the ODH reactor 4. Locations of the axial positions may depend on the operating temperature, the rate of the reaction, and the ethane selectivity, all of which impact the heat of reaction. By way of example only, if the cooled portion of the ethane feedstock was introduced into the ODH reactor 4 at one meter intervals, the cooled portion of the ethane feedstock may be introduced at a minimum of five locations of the ODH reactor 4. The cooled portion of the ethane feedstock may be supplied to the ODH reactors 4 by conventional techniques, such as by tubing, valves, etc., which are not described in detail herein. A sparger (not shown) may be present at each location to disperse and mix in the cooled portion of the ethane feedstock with the ethane feedstock 2 at the reaction temperature. Without being bound by any theory, it is believed that the resulting axial temperature profile of the ODH reactor 4 is such that the temperature rises across a section of the ODH reactor 4 and then is cooled by the cooled portion of the ethane feedstock. This process is repeated until the desired conversion of the ethane to ethylene is achieved. If the temperature in the ODH reactor 4 is plotted versus axial position in the ODH reactor 4, a sawtooth pattern of a slow increase in temperature followed by a rapid fall in temperature is observed. The energy for preheating the ethane feedstock 2 is reduced but there will be no recovery of energy at high temperature levels due to the direct mixing of the cooled portion of the ethane feedstock and the ethane feedstock 2 at the reaction temperature.

The indirect interbed cooling of the ODH reactors 4 may include reducing the temperature of the ethylene stream 8 exiting the ODH reactor 4. By way of example only, the ethylene stream 8 may be transported to the heat management unit 10, i.e., an external heat exchanger, through a thin walled internal head. Heat transfer between the ethylene stream 8 and the heat exchanger may reduce the temperature of the portion of the ethane feedstock while increasing the temperature of a boiler feed water stream of the heat exchanger. The boiler feed water stream may be used as an energy source, such as to produce steam. While the indirect cooling is more capital intensive than the direct cooling, i.e., the cold shot cooling, the indirect cooling provides energy recovery near the reaction temperature, resulting in minimal loss of energy. If the temperature in the ODH reactor 4 is plotted, a sawtooth pattern is observed with a temperature drop in the ethylene stream 8 as a result of the external heat exchange.

If the system 18 includes multiple tubular ODH reactors 4, such as 4 or 5 reactors in series, the system 18 may be isothermal and the conversion per ODH reactor 4 is about 25% ethane. Supercritical water may be used as the coolant. The temperatures in the ODH reactors 4 remain isothermal throughout the system 18 with the temperature level controlled by the coolant temperature and flow rate. Since the rate of reaction falls in the latter ODH reactors 4 of the system, the temperature level may be increased accordingly. There is no temperature exotherm, just merely an increase in level managed by the coolant due to the reduction in total pressure related to frictional pressure drop through the tubes of the ODH reactors 4.

As shown in FIG. 4, the membrane separation unit 12 may include at least one membrane 20 configured to separate the ethylene in the cooled ethylene stream 14 from the ethane, such as unreacted ethane, and other components of the cooled ethylene stream 14. While the membrane 20 is illustrated in FIG. 4 as being a flat sheet, the membrane 20 may be configured in other shapes. The size and thickness of the membrane 20 may be selected depending on the composition of the cooled ethylene stream 14. A single membrane 20 or multiple membranes (not shown) may be used in the membrane separation unit 12 to selectively remove the ethylene. If multiple membranes 20 are used, the membranes 20 may be the same or different materials and may be selected based on the composition of the cooled ethylene stream 14. In addition to the membrane 20, the membrane separation unit 12 may include conventional components, such as pumps, a vacuum, filters, tubing, valves, etc., which are not illustrated or described in detail herein. The cooled ethylene stream 14 may enter the bottom of the membrane separation unit 12 and purified ethylene 16 may exit the top of the membrane separation unit 12. However, the membrane separation unit 12 may be configured for a different direction of flow of the cooled ethylene stream 14. Multiple membrane separation units 12 may also be used in the system 18. The membrane separation unit 12 may optionally include another membrane (not shown) configured to separate the ethylene from other components in the cooled ethylene stream 14. By way of example only, the membrane separation unit 12 may be a swing membrane system. Since the membrane 20 selectively removes the ethylene, no distillation apparatus may be utilized to remove the ethylene, reducing capital costs associated with building the distillation apparatus.

The membrane 20 may be configured to separate the ethylene from the ethane with a high degree of selectivity. The membrane 20 may, thus, be highly selective for ethylene relative to unreacted ethane. The material of the membrane 20 may also be resistant to a temperature of the ethylene stream 8 exiting the ODH reactor, such as a temperature of less than or equal to about 100° C. The material of the membrane 20 may also be resistant to a pressure of the ethylene stream 8 exiting the ODH reactor. The material of the membrane 20 may provide reduced energy consumption, increased process intensification, and modularization. Since the ODH reaction according to embodiments of the disclosure is highly selective for the production of the ethylene with few byproducts, the material of the membrane 20 may be selected so that the ethylene permeates through the membrane 20 while the unreacted ethane does not permeate through the membrane 20. By way of example only, the membrane 20 may be a silver-based membrane, such as those sold under the OPTIPERM tradename, which is commercially available from Compact Membrane Systems (Newport, Del.).

The purified ethylene 16 may be recovered and stored in a vessel (not shown) for its desired use. If the purified ethylene 16 recovered from the membrane separation unit 12 is to be used as polymer grade ethylene, such as having an ethylene content of at least about 99.5% or greater or about 99.95% or greater, small scale distillation may optionally be utilized after the membrane separation to achieve the desired purity of the ethylene. However, the scope of any distillation is greatly reduced from conventional ethylene fractionators and may be eliminated completely with depending on the efficiency of the membrane separation. The unreacted ethane of the cooled ethylene stream 14 may be recovered and optionally reprocessed, such as by combination with the ethane feedstock 2. A purge may be utilized to combine the unreacted ethane of the cooled ethylene stream 14 with the ethane feedstock 2.

Since the selectivity of membranes for separating alkanes and alkenes is low, the high selectivity of the membrane 20 for separating the ethylene from the ethylene stream 8 was surprising. Since it is also known in the art that the ODH reaction is very exothermic, it was also surprising that the membrane 20 could be used to separate the ethylene from ethane because conventional membranes are not resistant to elevated temperatures.

Since the system 18 may include one or more ODH reactors 4 in combination with one or more membrane separation units 12, the system may provide a modular and tailorable system 18 configured to produce the purified ethylene 16. Since many components of the system 18 are modular, the system 18 according to embodiments of the disclosure may be a fraction of the cost and size of a conventional ethylene plant.

The method and system 18 according to embodiments of the disclosure may be incorporated into an existing olefins plant either as part of a full expansion, a modular addition, or as an add-on to an existing process. The amount of processing equipment may be minimal in terms of the number of unit operations and the extent of utilities needed. The purified ethylene 16 from the method and system 18 may be transported to a flare or to fuel gas headers of the existing olefins plant. The ethane feedstock 2 to be introduced to the ODH reactor 4 according to embodiments of the disclosure may be a fresh feedstock to the existing olefins plant or may be ethane recycled from the system 18. The purified ethylene 16 from the method and system 18 may be added to a quench tower of the existing olefins plant. Alternatively, the purified ethylene 16 from the method and system 18 may be added to the existing olefins plant at a later point, avoiding compression, chilling and demethanization. If the purified ethylene 16 from the system 18 is added directly to a deethanizer in the existing olefins plant, higher carbon compounds (C3+) may be processed along with the conventional stream in the existing olefins plant. Additionally if any acetylene is produced in the method and system 18, the acetylene may be hydrogenated and removed from the existing olefins plant using conventional equipment.

While the disclosure may be susceptible to various modifications and alternative forms, certain illustrative embodiments have been described by way of example in the drawings and have been described in detail herein. However, it should be understood that the disclosure is not intended to be limited to the particular forms disclosed. Rather, the disclosure is to cover all modifications, equivalents, and alternatives falling within the scope of the disclosure as defined by the following appended claims and their legal equivalents.

1. A method of producing ethylene, comprising:
subjecting a feedstock comprising ethane to oxidative dehydrogenation to produce an ethylene stream;
passing the ethylene stream through a membrane separation unit to separate the ethylene from unreacted ethane in the ethylene stream; and
recovering the ethylene from the membrane separation unit.

2. The method of claim 1, wherein subjecting a feedstock comprising ethane to oxidative dehydrogenation comprises subjecting the feedstock comprising the ethane and oxygen to the oxidative dehydrogenation.

3. The method of claim 1, wherein subjecting a feedstock comprising ethane to oxidative dehydrogenation comprises subjecting the feedstock comprising an excess of the ethane to the oxidative dehydrogenation.

4. The method of claim 1, wherein subjecting a feedstock comprising ethane to oxidative dehydrogenation comprises subjecting the feedstock comprising about 95% by volume of the ethane and about 5% by volume of oxygen to the oxidative dehydrogenation.

5. The method of claim 1, wherein subjecting a feedstock comprising ethane to oxidative dehydrogenation to produce an ethylene stream comprises subjecting the feedstock to the oxidative dehydrogenation to produce the ethylene stream comprising ethylene, water, a carbon oxide, acetic acid, and acetylene.

6. The method of claim 1, wherein subjecting a feedstock comprising ethane to oxidative dehydrogenation to produce an ethylene stream comprises subjecting the feedstock to the oxidative dehydrogenation to produce the ethylene stream comprising ethylene, water, a carbon oxide, acetic acid, acetylene, and unreacted ethane.

7. The method of claim 1, wherein passing the ethylene stream through a membrane separation unit to separate the ethylene from unreacted ethane in the ethylene stream comprises passing the ethylene stream through a silver-based membrane.

8. The method of claim 1, wherein recovering the ethylene from the membrane separation unit comprises recovering the ethylene having an ethylene content of at least about 90%.

9. The method of claim 1, wherein recovering the ethylene from the membrane separation unit comprises recovering the ethylene having an ethylene content of at least about 99%.

10. The method of claim 1, wherein recovering the ethylene from the membrane separation unit comprises recovering the ethylene having an ethylene content of at least about 99.5%.

11. The method of claim 1, further comprising recovering the unreacted ethane from the membrane separation unit.

12. A method of producing ethylene, comprising:
contacting ethane and oxygen in the presence of a mixed metal oxide catalyst to produce a stream comprising ethylene;
passing the stream comprising ethylene through a membrane separation unit to separate the ethylene from the stream comprising ethylene; and
recovering ethylene having an ethylene content of at least about 90% from the membrane separation unit.

13. The method of claim 12, wherein contacting ethane and oxygen in the presence of a mixed metal oxide catalyst to produce a stream comprising ethylene comprises contacting the ethane and the oxygen at a temperature of from about 300° C. to about 450° C. to produce the stream comprising ethylene.

14. The method of claim 12, wherein contacting ethane and oxygen in the presence of a mixed metal oxide catalyst to produce a stream comprising ethylene comprises contacting the ethane and the oxygen to produce the stream comprising ethylene at an ethane conversion of greater than about 90%.

15. The method of claim 12, wherein contacting ethane and oxygen in the presence of a mixed metal oxide catalyst to produce a stream comprising ethylene comprises contacting the ethane and the oxygen to produce the stream comprising ethylene at an ethylene selectivity of greater than about 95%.

16. The method of claim 12, further comprising cooling the stream comprising ethylene to a temperature of less than or equal to about 100° C. before passing the stream comprising ethylene through the membrane separation unit.

17. A system configured to produce ethylene, comprising:
at least one ODH reactor configured to convert ethane to ethylene;
a heat management unit coupled to the at least one ODH reactor and configured to reduce a temperature of the ethylene; and
at least one membrane separation unit comprising at least one membrane, the at least one membrane configured to separate the ethylene from unreacted ethane.

18. The system of claim 17, wherein the at least one ODH reactor comprises a tubular ODH reactor, a fixed bed ODH reactor, a fluidized bed ODH reactor, a packed bed ODH reactor, or combination thereof.

19. The system of claim 17, wherein the heat management unit comprises a supercritical water system coupled to the at least one ODH reactor.

20. The system of claim 17, wherein the at least one membrane comprises a silver-based membrane.

21. The system of claim 17, further comprising a distillator after the at least one membrane separation unit.

22. The method of claim 1, further comprising distilling the ethylene after recovering the ethylene from the membrane separation unit.
